(19) United States
(12) Patent Application Publication
(10) Pub. No.: US 2022/0139553 A1
(43) Pub. Date: May 5, 2022

(54) ANALYSIS SYSTEM, A METHOD AND A COMPUTER PROGRAM PRODUCT SUITABLE TO BE USED IN VETERINARY MEDICINE

(71) Applicant: GekkoVet Oy, Salo (FI)

(72) Inventors: Johanna MAJAMAA, Helsinki (FI); Pertti ORAKOSKI, Helsinki (FI); Anne VARJO, Tervalampi (FI); Jukka JYRÄSALO, Kotka (FI)

(21) Appl. No.: 17/090,183

(22) Filed: Nov. 5, 2020

(51) Int. Cl. G16H 50/20; G16H 10/20; G16H 10/60; G16H 50/70; G16H 70/20; G16H 40/67

(52) U.S. Cl. CPC G16H 50/20; G16H 10/20; G16H 10/60; A61B 5/7267; G16H 70/20; G16H 40/67; G16H 50/70

(57) ABSTRACT

The embodiments relate to a method and technical equipment, where the method comprises receiving over a data transfer network information comprising at least a set of symptoms concerning a subject; executing a machine learning algorithm to determine one or more conditions based on said received information and to determine instructions for improving said condition; transmitting at least one of the instructions to a client apparatus over a data transfer network; continuously receiving information on the subject, which information relates to an outcome of the applied instructions on the subject; and training the machine learning algorithm with the received information to update the parameters being used for determining the condition and the instructions.

### TECHNICAL FIELD

The present solution generally relates to an analysis system suitable to be used in veterinary medicine. In particular, the solution relates to an self-learning system for providing such analysis.

### BACKGROUND

Pets and domestic animals have been part of people's life for a long time. Today, increasingly more attention is paid to pets' and animals' health and well-being. Since animals are not able to indicate their feelings nor e.g. pain unambiguously, it is a responsibility of an owner to monitor the animal and to interpret how a recovery from e.g. an illness is proceeding. In addition, this requires regular control visits to a veterinarian, where observations on animal's recovery are gone through and if necessary, the diagnose may be corrected.

The present embodiments provide a solution by means of which interaction between a pet owner and a veterinarian can be greatly improved, and by means of which more accurate diagnoses and treatment plans may be given.

### SUMMARY

The scope of protection sought for various embodiments of the invention is set out by the independent claims. The embodiments and features, if any, described in this specification that do not fall under the scope of the independent claims are to be interpreted as examples useful for understanding various embodiments of the invention.

Various aspects include a method, an apparatus and a computer readable medium comprising a computer program stored therein, which are characterized by what is stated in the independent claims. Various embodiments are disclosed in the dependent claims.

According to a first aspect, there is provided a method comprising receiving over a data transfer network information comprising at least a set of symptoms concerning a subject; executing a machine learning algorithm to determine one or more conditions based on said received information and to determine instructions for improving said condition; transmitting at least one of the instructions to a client apparatus over a data transfer network; continuously receiving information on the subject, which information relates to an outcome of the applied instructions on the subject; and training the machine learning algorithm with the received information to update the parameters being used for determining the condition and the guidance.

According to a second aspect, there is provided an apparatus comprising at least one processor, memory including computer program code, the memory and the computer program code configured to, with the at least one processor, cause the apparatus to perform at least the following: receive over a data transfer network information comprising at least a set of symptoms concerning a subject; execute a machine learning algorithm to determine one or more conditions based on said received information and to determine instructions for improving said condition; transmit at least one of the instructions to a client apparatus over a data transfer network; continuously receive information on the subject, which information relates to an outcome of the applied instructions on the subject; and train the machine learning algorithm with the received information to update the parameters being used for determining the condition and the instructions.

According to a third aspect, there is provided a computer program product comprising computer program code configured to, when executed on at least one processor, cause an apparatus or a system to: receive over a data transfer network information comprising at least a set of symptoms concerning a subject; execute a machine learning algorithm to determine one or more conditions based on said received information and to determine instructions for improving said condition; transmit at least one of the instructions to a client apparatus over a data transfer network; continuously receive information on the subject, which information relates to an outcome of the applied instructions on the subject; and train the machine learning algorithm with the received information to update the parameters being used for determining the condition and the instructions.

According to an embodiment, the subject is an animal.

According to an embodiment, the information related to the outcome is stored in a database comprising data on veterinary medicine.

According to an embodiment, the information comprising at least a set of symptoms is received from a client apparatus, said set of symptoms comprising one or more symptoms being selected by a user of the client apparatus.

According to an embodiment, the information comprising at least a set of symptoms on the subject is received from a wearable device of the subject.

According to an embodiment, control instructions are generated based on the instructions for improving the condition, wherein said control instructions are transmitted to an external device in order to control the operations of the external device.

According to an embodiment, the information on the outcome of the applied instructions on the subject is continuously received from a client device.

According to an embodiment, the information on the outcome of the applied instructions on the subject is continuously received from the external device whose operation is being controlled.

### DESCRIPTION OF THE DRAWINGS

In the following, various embodiments will be described in more detail with reference to the appended drawings, in which

FIG. 1 shows a simplified example of a system according to an embodiment;

FIG. 2 shows examples of a client device and a veterinarian device;

FIG. 3 shows an embodiment of a method from client device's point of view;

FIG. 4 shows an embodiment of a method from the point of view of the analysis tool;

FIG. 5 illustrates an example of a machine learning algorithm model; and

FIG. 6 is a flowchart illustrating a method according to an embodiment.

### DESCRIPTION OF EXAMPLE EMBODIMENTS

The present solution is targeted to an analysis system for veterinary diseases and animal health-care. The system provides a pet owner a comprehensive solution for taking care of the pet in situations where the pet is suffering from health-threatening and/or health-affecting symptoms. In addition, the analysis tool provides a veterinarian, i.e. a vet, solution for improved diagnoses. Even though the present embodiments are discussed by using animals and veterinary diseases as example, the technical solution behind the application can be utilized in other environments as well.

The analysis system being disclosed can be implemented as a hybrid solution that combines both virtual and non-virtual, i.e. physical, consultation. However, it is possible that by using and developing the present analysis system, the portion of the non-virtual consultation compared to the virtual consultation may decrease. The analysis tool is usable online, but also offline use is possible, when the necessary data has been locally stored in the client or the veterinarian devices.

The analysis system may be implemented as two computer programs; one for a client device, i.e. a pet owner, and another for a veterinarian device. The application for the veterinarians is also called “analysis tool”, since it provides intelligent veterinary services to a client. The client application and the veterinarian application are able to operate independently with the medical data being stored in a medical database. However, when desired, the connection between the computer applications can be formed so that more comprehensive medical data can be provided to the client, but also that information on symptoms and healing process can be provided to the vet through the application.

The present embodiments rely on real-life and real-time data on pet's healing process in relation to a given treatment plan. Such a data can be provided to an analysis tool comprising a machine learning algorithm, but also to be shown on a user interface of the veterinarian application. The present embodiments are based on receiving information on a condition of a subject; determining instructions by means of which the condition can be improved; receiving feedback on an outcome of the applied instruction on the subject.

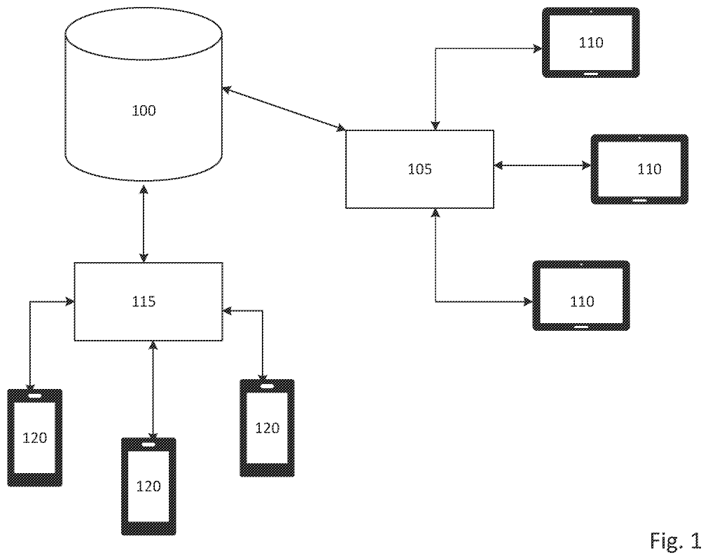

An example of the present solution has been illustrated in FIG. 1. FIG. 1 shows a database 100 storing data that is to be used for determining the instructions to be used for improving the condition of a subject. In the example of veterinary application, the database stores medical data for animals. The content of the database 100 can be viewed by veterinarian devices 110 or client devices 120. However, it is appreciated that the view being provided to client application 115, is more limited than the view being provided to the veterinarian application 105. This means that the medical data stored in the database 100 may be classified into data that is accessible by clients and into data that is accessible by veterinarians.

According to an embodiment, the client application is a part of the veterinarian software. This means that the veterinarian software comprises a client application module to be downloadable to client devices. It is appreciated that term “client application” refers to both an independent client software, but also to a client module provided by a veterinarian software.

When the data stored in the database 100 is for the veterinary application, the data comprises veterinary literature on animal diseases, their symptoms, medication, treatment etc. In addition, the data stored in the database 100 comprises data concerning real-life observations and feedback gathered from animals going through a certain treatment plan for a certain illness. Thus, the database stores structured data on animal patients. The data comprises combinations of chosen symptoms, values, diagnoses and chosen treatments. In addition, outcomes of chosen treatments may be stored. All the data being stored in the database is anonymized and available for data extracts only on aggregated level. The benefit of collecting and analyzing real-world data is the possibility to evaluate chosen therapies and their outcomes in broad, heterogenous populations. This has not been possible in randomized clinical trials with strict inclusion and exclusion criteria. Thus, the database acts as a global real-world veterinary medicine database, where the real-world data can be utilized e.g. for guideline formation or for a decision support tool in clinical practice and generally develop veterinary medicine further.

For using the system, the user of the client device may input various symptoms relating to his/her animal to the client application. In addition or alternatively, symptoms of an animal and/or other data concerning the animal can be automatically obtained from a wearable device of an animal. Examples of such wearables are activity collar, activity harness, microchip etc. For example, an activity collar may provide data on the steps being taken or the length of the walk.

The client application comprises a user interface view that may have fields into which a user can enter keywords and search for a symptoms; and/or pull-down menus from which the user can select suitable symptoms. The symptoms being input by the user are received by an analysis algorithm as input, and matched at the database 100 to various diseases, and a list of possible diseases is returned to the client application as an output—to be shown on a user interface of the application.

A vet may use the veterinarian application 105 aimed for the veterinarian device similarly. The vet may input more detailed data (i.e. symptoms, laboratory results, etc.) to the user interface of the application 105, as a response to which, a list of possible diseases with more detailed instructions (e.g. medication, treatment plan, a request for more data) is returned to the veterinarian application 105, to be shown on a user interface.

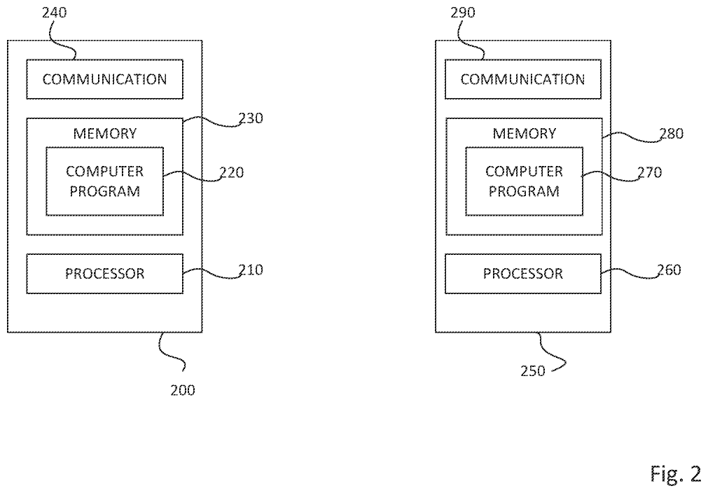

FIG. 2 illustrates the veterinarian device 200 and the client device 250. Both devices 200, 250 comprise a computer program 220, 270 being stored in a memory 230, 280 of the device 200, 250. The computer program 220 comprises computer code, which—when executed by a processor 210 of the veterinarian device 200—causes the veterinarian device 200 to act as an analysis tool for veterinarians, i.e. veterinarian application. The analysis tool also acts as a database (shown in FIG. 1) for the medical data. The computer program 270 for a client device 250 comprises computer code, which—when executed by a processor 260 of the client device 250—causes the client device 250 to act as a client for the analysis tool. Therefore, the computer program 270 is targeted for consumers, i.e. pet owners, while the computer program 220 is targeted for professionals, i.e. veterinarians. Both devices 200, 250 comprise communication means 240, 289 by means of which communication between devices 200, 250 and data transfer over a network can be accomplished.

As said, the client and veterinarian applications operate with a database comprising medical data being obtained from veterinary literature. The database also comprises recovery or healing data being gathered from animal patients. In addition the veterinarian application, i.e. the analysis tool, comprises a machine learning algorithm to operate with the data being stored in the database. The purpose of the machine learning algorithm is to receive a set of preselected symptoms as an input, determine a set of diseases based on the preselected symptoms, and to generate one or more treatment plan drafts for the set of diseases as an output. In addition, the purpose of the machine learning algorithm is to receive recovery, i.e. healing, data and to update its parameters accordingly to determine suitable diseases and treatment plans more accurately. The veterinarian application is configured to display the treatment plan drafts to a user of the veterinarian application, so that the user (i.e. the vet) can select—an optionally further modify—one of the treatment plans for the animal patient.

Thus, the machine learning algorithm is continuously learning from the data that is obtained from the pet owner. Such a data comprises—not only the symptoms being input at first—but data concerning the healing process of the pet. With such continuous training, the operation of the machine learning algorithm is improved because the parameters affecting to the output of the machine learning algorithm are adjusted based on the feedback from the pet's owner.

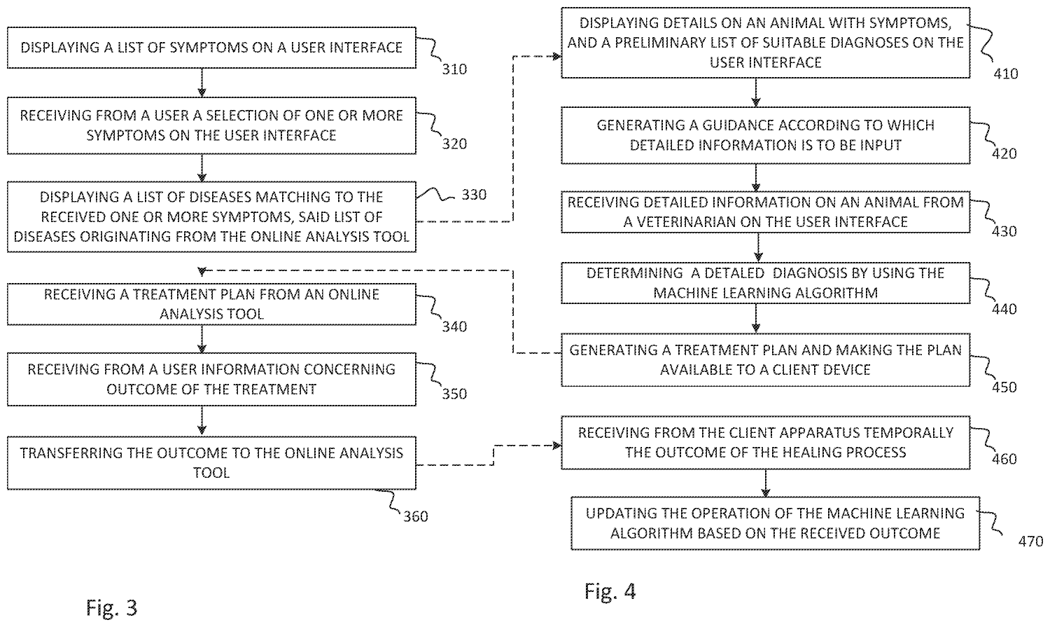

The operation of the analysis tool is discussed next. At first, process steps from the point of view of the client device 250 are discussed. Then process steps from the point of view of the veterinarian device 200 are discussed. These steps are also illustrated in flowcharts shown in FIGS. 3 and 4, respectively.

FIG. 3 illustrates a method implemented at the client device 250 according to an embodiment. In this embodiment the method comprises the following steps:
displaying 310 on a user interface of the computer program a pre-defined list of symptoms for an animal. The predefined list of symptoms originate from an analysis tool, to which the client device has a connection;
receiving 320 from a user a selection of one of more symptoms appearing in the predefined list of symptoms, which symptoms the user has noticed from the animal, wherein the selection is made on a user interface of the computer program. Instead or in addition, symptoms and features relating to an animal may be received automatically from a wearable of the animal;
as a response to selected symptoms, displaying 330 a list of possible diagnoses, i.e. diseases and optionally also recommendation on suitable veterinary service and/or a care instructions. The list of possible diagnoses is generated by the analysis tool, and provided to the user interface of the client device over the communication network;
after a possible (physical) visit on a veterinarian, a detailed treatment plan is received 340 from the analysis tool through to be automatically stored in the health diary of the animal in the client device. In addition, the treatment plan is displayable on a user interface of the client application. The treatment plan and the care instructions enable the user to carry out the correct treatment for his/her pet. In addition, certain parts of the treatment plan may be automatically used for controlling external device being used by the animal patient. For example, due to the treatment plan, a treadmill, an underwater treadmill, an activity collar, an automatic dog feeder etc. can be controlled to give the suitable training for the animal patient. In addition, any of the external device may provide feedback data to the analysis tool as an input on the progress of the treatment plan. An example of this is an activity collar, which—according to a received control signal—monitors whether a certain activity level has been achieved, and makes and alarm on that. This alarm may automatically transmitted to a pet owner's device but also (or in addition) to analysis tool;
receiving 350 temporally information from a user through a user interface of the client application, wherein the information concerns at least the outcome of the treatment and the healing process. Thus the information may comprise experiments on how the medication or other treatment operations have succeeded; observations on the current condition of the animal (worse—better); information on whether any adverse effects have been noticed, etc.;
transferring 360 the received outcome to the analysis tool;
continuing two previous steps until the veterinarian notices that the animal has healed, or until a predefined time has lapsed, or until a predefined number of feedback queries has been made; or until the veterinarian requests the user to bring the animal to the veterinarian, whereafter a new treatment plan may be received.

In the previous method, the pet is to be taken to the vet after a list of possible diagnoses has been received (i.e. after step 330). At least at that point, i.e. at the vet, the data—at least details on the animal and the selected symptoms—in the client application is made available to the computer program of the veterinarian.

Thereupon, according to an embodiment, the method at the veterinarian's device follows the following steps, which are also illustrated in FIG. 4:
receiving from a client application information comprising at least details on the animal and selected symptoms. The data is received over a communication network being formed between the client application and the analysis tool of the veterinarian;
generating a preliminary list of suitable diagnoses based on the received symptoms, and displaying 410 the list as well as received details on the animal and selected symptoms on the user interface of the analysis tool;
generating 420 a guidance, wherein the guidance is derived from a data relating to the generated list of suitable diagnoses and wherein the purpose of the guidance is to guide the veterinarian to define other clinical symptoms relating to the animal, to take laboratory tests etc.; and displaying the guidance on the user interface of the veterinarian application;
receiving 430 via a user interface of the analysis tool information on the animal, which information comprises clinical and/or test results (e.g. laboratory values) being made according to the guidance and which information is input by the veterinarian;
determining 440 a more detailed diagnosis for the animal based on the data that have been received;
displaying the detailed diagnosis on the user interface of the analysis tool with treatment options;
creating a treatment plan 450, i.e. instructions, comprising at least the diagnosis and treatment options, but optionally also symptoms and/or results;
transmitting the treatment plan to the client application;
receiving 460 regularly outcome of the healing process from the client application;
updating 470 the operation of the machine learning algorithm of the analysis tool based on the received outcome.

The outcome data on the healing process is used to train the machine learning algorithm of the analysis tool to improve the operation of the algorithm on different diagnoses and to select the most efficient treatment plans. Due to the learning, the analysis tool may also affect to the possible diagnoses provided to the client application.

As discussed above, the present embodiments enable enriching literary-based veterinary data being stored in a database with actual experience-based and real-world knowledge which is obtained by monitoring the healing process of an animal and/or feedback data being provided by wearables of the animal patient during the healing process. Therefore, one purpose of the present embodiments is to generate new veterinary data by fusing the experience-based data with the literary-based data.

Prior the present solution, any information concerning the healing process of an animal is received from the pet owner during a visit at the vet. Such an information may or may not impact the diagnosis, depending on the level of interaction between the vet and the pet owner. In addition, sometimes the pet owner is not able to meet the vet, whereupon s/he has limited capabilities to discuss the symptoms with the vet. Consequently, the diagnosis and treatments may vary a lot, and may not be comparable between animals.

The present embodiments improve the interaction between the pet owner and the vet, since symptoms have been standardized throughout the system and may be given to the analysis tool in such standardized format. Thus, any symptom being input has direct effect on the results being deciphered by the machine learning algorithm of the analysis tool. This means that the input/information from the pet owners has the necessary impact when vets are seeking correct diagnoses. Such a standardization in the veterinary field has no been existed before, since there has not been means for generating one. There are no governmental or official worldwide standards for symptoms or diseases in the veterinary field. Thus, the present analysis tool gives a standard not only for veterinarians but also for pet owners, and connects these in a new way.

The connection between the applications works in both directions. When the vet selects the treatment plan for the pet from the suggested treatment plans, the selection has a direct effect on the client application, because the selected treatment plan is transferred automatically to the client application and saved on pet owners client application's health diary.

Prior the present solution, getting the healing information of the patient, i.e. the result of the treatment process (whether the animal has cured or how well the treatment has worked) is hard or almost impossible to get. Owners do not report back voluntarily in most cases and vets do not have time to track them. By the present solution, the attending vet can monitor the healing process as the pet owner inputs data to the application to be forwarded to the analysis tool. Alternatively, or in addition the information on the healing process can be transmitted directly to the machine learning algorithm of the analysis tool, in order to get an updated recommendation on the treatment. The recommendation may be to continue with the current treatment plan, or to change medication or to increase exercise periods etc. The recommendation can be transmitted back to the client application, and/or to the veterinarian application for verification.

Since the data being used for continuously training the machine learning algorithm is increased due to real animal patient information, the operation of the machine learning algorithm is greatly improved. Such a data cannot be reasonably and/or correctly gathered without the present solution. Thus the unique combination of the veterinary scientific literature and the real patient information causes the machine learning algorithm to generate outputs, which take both of these dimensions into account.

Therefore, one of the aspects of the present solution is in gathering data. The present embodiments provide means to collect data on patients, which data relates to a healing process of the patient, i.e. to an outcome of the treatment plan. The data being collected via a client application is stored in a veterinary database to be available when another treatment plan is being created for a patient suffering from similar symptoms.

As an example, the machine learning algorithm can comprises several steps:
at first a first set of symptoms is received as input;
a set of diagnoses is determined based on the set of symptoms;
a treatment plan recommendations are provided as output.

One of the treatment plans is selected to be used for the animal patient. Then, the machine learning algorithm is able to perform the following:
receiving data concerning an outcome of the treatment plan relating to the symptoms frequently;
updating the set of diagnoses based on the received data;
updating the treatment plan recommendations based on the received data;
continuing the steps until it has been determined that the patient has been healed.

It is appreciated that the amount of data used for training the machine learning algorithm increases by every patient.

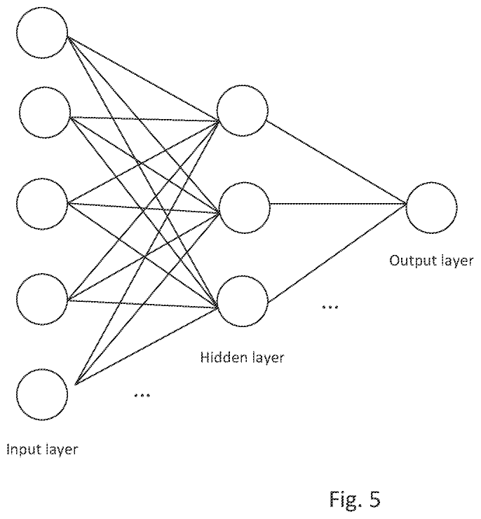

An example of a machine learning algorithm model is illustrated in FIG. 5. In here the machine learning model is a neural network. However, it is appreciated that any machine learning algorithm (e.g. deep learning, regression method) can be used instead. The neural network comprises an input layer, one or more hidden layers, and an output layer. The input layer receives data concerning one or more of the following: human detectable symptoms; automatically obtained (e.g. from one or more sensors being attached to the animal) symptoms/properties; animal-related information on e.g. species, breed, age, etc. The one or more hidden layers uses constant data stored in the database, real-life data gathered from patients, laboratory tests, physical examination performed by the vet, X-ray-imaging, ultrasonography, preliminary symptoms, results and findings, etc. in order to make determination on suitable disease(s) and treatment plans. The output layer outputs one or more diseases and/or treatment plans to be used on the animal patient. The output may also be used for controlling external devices, such as an activity collar, a treadmill, an automatic dog feeder etc. These external devices may also provide back data concerning the healing process of the animal, due to which the operation of the machine learning algorithm may be improved.

At the starting point, the operation of the machine learning algorithm is based on scientific research and latest veterinary medicine literature. However, when more and more veterinarians use the analysis tool, the more real-world data worldwide will be accumulated into the database of the analysis tool. The present solution has been designed to collect and analyze from the beginning and it has a solid technical data platform to perform complex analyzes. Once enough data exist in the database, the machine learning algorithms can be utilized to determine various correlations between certain variables (such as breed, age, symptoms or disease) or to look at the regional population deviation, to mention only few as examples.

The present embodiments are further discussed with reference to an example of a use case. A dog, Giant Schnauzer, age 7, has been lethargic, has gained weight and lost hair a lot, and has infection on skin and hairless areas. Owner selects these symptoms from the client application, and receives one or more possible diseases as a response. One of the diseases is hypothyroidism. The owner takes the dog to the vet, who examines the dog and by using the analysis tool, the vet is guided to look for other clinical signs typical of hypothyroidism, such as slow heart rate. The analysis tool also lists typical changes in laboratory values, and the vet takes a blood test, as a result of which determines low thyroxin level. The dog is diagnosed of hypothyroidism and the analysis tool shows treatment plan options for the vet including levothyroxine medication on the dosage level of 0.02-0.04 mg/kg per day. The vet chooses 0.04 mg/kg per day for the dog, and the treatment plan is transferred to owner's client application. The owner gives the mediation as instructed, and receives a notification after one week to report outcome of the treatment plan. The owner inputs data concerning the outcome and reports that medication has been given without problems and the previous symptoms have disappeared. In addition to these, the owner informs on an adverse effect, that the dog has been very nervous and hyperactive. After receiving the data from the owner, the vet updates the treatment plan so that the dosage of the medicine is lowered to 0.02 mg/kg per day, and the updated treatment plan is automatically transferred to the client application to be stored. After one week, the owner receives another notification, and reports back that the symptoms are still cured, and the dog is acting normal again. The vet receives the information and set a new notification to be sent after one month to control again.

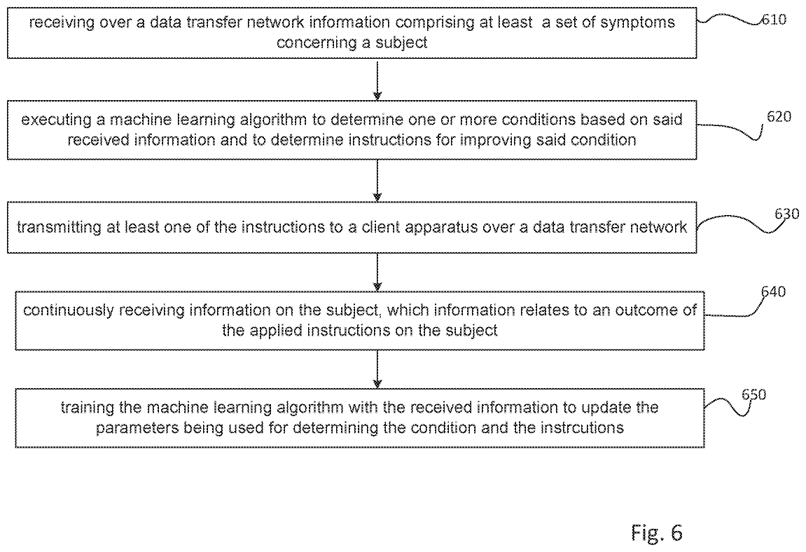

In previous, embodiments for generating an analysis tool for veterinarian and their patients has been disclosed. The analysis too perform a method at the veterinarian device, of which an embodiment is shown in FIG. 6.

The method according to an embodiment is shown in FIG. 6. The method generally comprises receiving 610 over a data transfer network information comprising at least a set of symptoms concerning a subject; executing 620 a machine learning algorithm to determine one or more conditions based on said received information and to determine instructions for improving said condition; transmitting 630 at least one of the instructions to a client apparatus over a data transfer network; continuously receiving 640 information on the subject, which information relates to an outcome of the applied instructions on the subject; and training 650 the machine learning algorithm with the received information to update the parameters being used for determining the condition and the instructions. Each of the steps can be implemented by a respective module of a computer system.

An apparatus according to an embodiment comprises means for receiving over a data transfer network information comprising at least a set of symptoms; means for executing a machine learning algorithm to determine one or more conditions based on said received information and to determine instructions for improving said condition; means for transmitting at least one of the instructions to a client apparatus over a data transfer network; means for continuously receiving information on the subject, which information relates to an outcome of the applied instructions on the subject; and means for training the machine learning algorithm with the received information to update the parameters being used for determining the condition and the guidance. The means comprises at least one processor, and a memory including a computer program code, wherein the processor may further comprise processor circuitry. The memory and the computer program code are configured to, with the at least one processor, cause the apparatus to perform the method of FIG. 6 according to various embodiments.

The various embodiments may provide advantages. For example, the present embodiments provide a solution that interconnects veterinary knowledge derived from accepted literature with real-life outcome data. In addition, the present system provides an interactive and standardized way of transferring data between vets and pet owners by providing a digital solution to be used for interaction. This a great improvement compared to the prior solutions, which relate only to human interaction and how they communicate symptoms, diagnose and treatments back and forth.

The various embodiments can be implemented with the help of computer program code that resides in a memory and causes the relevant apparatuses to carry out the method. For example, a client device may comprise circuitry and electronics for handling, receiving and transmitting data, computer program code in a memory, and a processor that, when running the computer program code, causes the device to carry out the features of an embodiment. Yet further, a network device like a server may comprise circuitry and electronics for handling, receiving and transmitting data, computer program code in a memory, and a processor that, when running the computer program code, causes the network device to carry out the features of an embodiment.

A computer program product according to an embodiment can be embodied on a non-transitory computer readable medium. According to another embodiment, the computer program product can be downloaded over a network in a data packet.

If desired, the different functions discussed herein may be performed in a different order and/or concurrently with other. Furthermore, if desired, one or more of the above-described functions and embodiments may be optional or may be combined.

Although various aspects of the embodiments are set out in the independent claims, other aspects comprise other combinations of features from the described embodiments and/or the dependent claims with the features of the independent claims, and not solely the combinations explicitly set out in the claims.

It is also noted herein that while the above describes example embodiments, these descriptions should not be viewed in a limiting sense. Rather, there are several variations and modifications, which may be made without departing from the scope of the present disclosure as, defined in the appended claims.

1. A method, comprising:
receiving over a data transfer network information comprising at least a set of symptoms concerning a subject;
executing a machine learning algorithm to determine one or more conditions based on said received information and to determine instructions for improving said condition;
transmitting at least one of the instructions to a client apparatus over data transfer network;
continuously receiving information on the subject, which information relates to an outcome of the applied instructions on the subject; and
training the machine learning algorithm with the received information to update the parameters being used for determining the condition and the instructions.

2. The method according to claim 1, wherein the subject is an animal.

3. The method according to claim 2, further comprising storing the information relating to the outcome in a database comprising data on veterinary medicine.

4. The method according to claim 1, wherein information comprising at least a set of symptoms is received from the client apparatus, wherein said set of symptoms comprises one or more symptoms being selected by a user of the client apparatus.

5. The method according to claim 1, wherein information comprising at least a set of symptoms is received from a wearable device of the subject.

6. The method according to claim 1, further comprising generating control instructions based on the instructions for improving the condition, wherein said control instructions are transmitted to an external device in order to control the operations of the external device.

7. The method according to claim 1, wherein the information on the outcome of the applied instructions on the subject is continuously received from a client device.

8. The method according to claim 6, wherein the information on the outcome of the applied instructions on the subject is continuously received from the external device whose operation is being controlled.

9. An apparatus comprising at least one processor, memory including computer program code, the memory and the computer program code configured to, with the at least one processor, cause the apparatus to perform at least the following:
receive over a data transfer network information comprising at least a set of symptoms concerning a subject;
execute a machine learning algorithm to determine one or more conditions based on said received information and to determine instructions for improving said condition;
transmit at least one of the instructions to a client apparatus over data transfer network;
continuously receive information on the subject, which information relates to an outcome of the applied instructions on the subject; and
train the machine learning algorithm with the received information to update the parameters being used for determining the condition and the instructions.

10. The apparatus according to claim 9, wherein the subject is an animal.

11. The apparatus according to claim 10, further being configured to store the information relating to the outcome in a database comprising data on veterinary medicine.

12. The apparatus according to claim 9, further being configured to receive information comprising a set of symptoms from a client apparatus, said set of symptoms comprising one or more symptoms being selected by a user of the client apparatus.

13. The apparatus according to claim 9, further being configured to receive information comprising a set of symptoms from a wearable device of the subject.

14. The apparatus according to claim 9, further being configured to generate control instructions based on the instructions for improving the condition, wherein said control instructions are transmitted to an external device in order to control the operations of the external device.

15. The apparatus according to claim 9, wherein the information on the outcome of the applied instructions on the subject is continuously received from a client device.

16. The apparatus according to claim 14, wherein the information on the outcome of the applied instructions on the subject is continuously received from the external device whose operation is being controlled.

17. A computer program product comprising computer program code configured to, when executed on at least one processor, cause an apparatus or a system to:
receive over a data transfer network information comprising at least a set of symptoms concerning a subject;
execute a machine learning algorithm to determine one or more conditions based on said received information and to determine instructions for improving said condition;
transmit at least one of the instructions to a client apparatus over a data transfer network;
continuously receive information on the subject, which information relates to an outcome of the applied instructions on the subject; and
train the machine learning algorithm with the received information to update the parameters being used for determining the condition and the instructions.
